(19) United States
(12) Patent Application Publication
(10) Pub. No.: US 2010/0305403 A1
(43) Pub. Date: Dec. 2, 2010

(54) Sheath Assembly

(75) Inventors: James Alastair Louis Bartlett, Gloucester (GB); Gary Victor Louis Warner, Wiltshire (GB); Neil Brian Cant, Gloucester (GB)

Correspondence Address: BARNES & THORNBURG LLP, P.O. BOX 2786, CHICAGO, IL, 60690-2786, US

(73) Assignee: Q PARK MEDICAL LIMITED, Swindon, Wiltshire (GB)

(21) Appl. No.: 12/677,986

(22) PCT Filed: Sep. 16, 2008
(86) PCT No.: PCT/GB08/03126

§ 371 Date: Mar. 12, 2010

(30) Foreign Application Priority Data
Sep. 17, 2007 (GB) 0718093.8

(51) Int. Cl. A61B 1/00

(52) U.S. Cl. 600/125

(57) ABSTRACT

A sheath assembly for protecting an endoscope insertion tube projecting from an endoscope head. The sheath assembly comprises: a resilient elongate tube comprising a bore for receiving the endoscope insertion tube; a resilient fitting for gripping the endoscope head; and a connector comprising a proximal end attached to the resilient fitting and a distal end attached to the tube. The connector and the resilient fitting are formed from different materials, the material forming the connector having a higher elastic modulus than the material forming the resilient fitting. The higher stiffness of the connector enables it to transmit axial forces evenly and efficiently to the fitting. The connector has a pair of laterally extending wings which provide a convenient and ergonomic point to grip the connector, making it easier to fit the assembly onto the endoscope head. The wings also act as finger guards which keep the surgeon's fingers well away from the proximal end of the fitting, reducing the chance of cross-infection with the endoscope head.

### FIELD OF THE INVENTION

The present invention relates to a sheath assembly for protecting an endoscope insertion tube projecting from an endoscope head.

### BACKGROUND OF THE INVENTION

A conventional sheath assembly is described in WO 02/07787 A2. The sheath assembly comprises an elastic elongate tube comprising a bore for receiving an endoscope insertion tube; and a resilient fitting for gripping the endoscope head. The resilient fitting is attached to the proximal end of the tube.

A problem with this arrangement is that the fitting must be sufficiently flexible to enable it to expand to accommodate the endoscope head, whilst also being sufficiently rigid to enable it to be gripped and pushed onto the endoscope head. Also, the elastic elongate tube is stretched over the fitting, and this can cause the fitting to contract and obstruct the passage of the endoscope insertion tube into the assembly. Furthermore, because the assembly is fitted onto an endoscope by gripping the fitting towards its proximal end, it is likely that the fingers will contact the endoscope head and cause cross-infection.

### SUMMARY OF THE INVENTION

A first aspect of the invention provides a sheath assembly for protecting an endoscope insertion tube projecting from an endoscope head, the sheath assembly comprising:
a resilient elongate tube comprising a bore for receiving the endoscope insertion tube;
a resilient fitting for gripping the endoscope head; and
a connector comprising a proximal end attached to the resilient fitting and a distal end attached to the tube,
   wherein the connector and the resilient fitting are formed from different materials, the material forming the connector having a higher elastic modulus than the material forming the resilient fitting.

A further aspect of the invention provides a method of protecting an endoscope insertion tube projecting from an endoscope head using the assembly of the first aspect, the method comprising:
inserting the endoscope insertion tube into the bore of the resilient elongate tube;
gripping the connector; and
applying a force to the connector so as to push the resilient fitting onto the endoscope head.

By forming the resilient fitting from a low modulus material, it is sufficiently flexible to enable it to easily be fitted to the endoscope head. The higher modulus of the connector enables it to be gripped easily, transmit axial forces evenly and efficiently to the fitting without bending or otherwise distorting, and retain its shape to avoid obstructing the endoscope insertion tube.

The resilient fitting and/or the connector may each be formed from multiple pieces. However for manufacturing simplicity they are preferably formed from a single piece of material.

Preferably the connector comprises one or more laterally extending projections, such as an annular flange or a pair of wings extending laterally from opposite sides of the connector. This enables the projection(s) to act effectively as a finger guard, minimising the risk of cross-infection of the endoscope head. Also, force can be applied to the projection(s) to push the resilient fitting onto the endoscope head. Typically the or each projection protrudes laterally from the body of the connector by more than 5 mm, and more preferably by more than 10 mm.

The distal end of the connector may be received in a proximal end of the bore of the tube, or vice versa. In the former case the high elastic modulus of the connector prevents it from contracting laterally if the tube is stretched over it. Thus the bore through the connector remains open and does not obstruct the passage of the endoscope insertion tube.

Preferably the distal end of the connector has an outer surface which engages an inner surface of the tube and is substantially non-tapered. This lack of taper minimises the forces between the two parts, and hence minimises distortion of the connector.

The resilient fitting and the connector may be fitted together in a number of ways. Typically the connector comprises a bore, and the resilient fitting comprises a distal end which is received in a proximal end of the bore of the connector. Alternatively the parts may be butted against each other, end to end, and bonded. Alternatively the distal end of the resilient fitting may have a bore which receives the proximal end of the connector.

Typically the connector and resilient fitting are bonded together, for instance by an adhesive or by a solvent which causes them to fuse together.

Typically the resilient fitting comprises a bore for receiving and gripping the endoscope head. The bore may be tapered or non-tapered, and may have one or more gripping projections such as annular rings.

Typically the resilient fitting comprises a bore with walls defined by a resilient material, such as an elastomer or soft PVC.

Typically the tube comprises a slip agent, either added as a discrete layer to its inner and/or outer surface, or an integral part of the material forming the tube.

The tube may be extruded, or non-extruded for instance as described in WO 02/07787 A2.

Preferably the assembly is adapted such that when the resilient fitting is fitted onto the endoscope head, the elastic elongate tube stretches axially and presses against a distal end of the endoscope insertion tube.

A further aspect of the invention provides a sheath assembly for protecting an endoscope insertion tube projecting from an endoscope head, the sheath assembly comprising:
a resilient elongate tube comprising a bore for receiving the endoscope insertion tube; and
a connection device attached to a proximal end of the tube, the connection device comprising a resilient fitting for gripping the endoscope head; and a pair of wings extending laterally from opposite sides of the connection device.

A further aspect of the invention provides a method of protecting an endoscope insertion tube projecting from an endoscope head using the assembly of the preceding aspect, the method comprising:
inserting the endoscope insertion tube into the bore of the resilient elongate tube; and
applying a force to the laterally extending wings so as to push the resilient fitting onto the endoscope head.

The connection device may be formed from a single piece, and may even be formed integrally with the resilient tube. Alternatively the connection device may be formed from two or more pieces (for instance a separate resilient fitting and connector as described in the first aspect of the invention).

Preferably the wings are positioned towards the distal end of the connection device, that is closer to its distal end than its proximal end. This enables the wings to act effectively as finger guards, minimising the risk of cross-infection of the endoscope head. Also, force can be applied to the wings to push the resilient fitting onto the endoscope head.

### BRIEF DESCRIPTION OF THE DRAWINGS

Embodiments of the invention will now be described with reference to the accompanying drawings, in which:

FIG. 1 is an exploded view of a sheath assembly;

FIG. 2 is a cross-section showing the proximal end of the assembly;

FIG. 3 is an enlarged cross-section showing the attachment between the fitting, the connector and the tube;

FIG. 4 is a cross-section showing the distal end of the assembly; and

FIG. 5 shows the assembly fitted onto an endoscope.

### DETAILED DESCRIPTION OF EMBODIMENT(S)

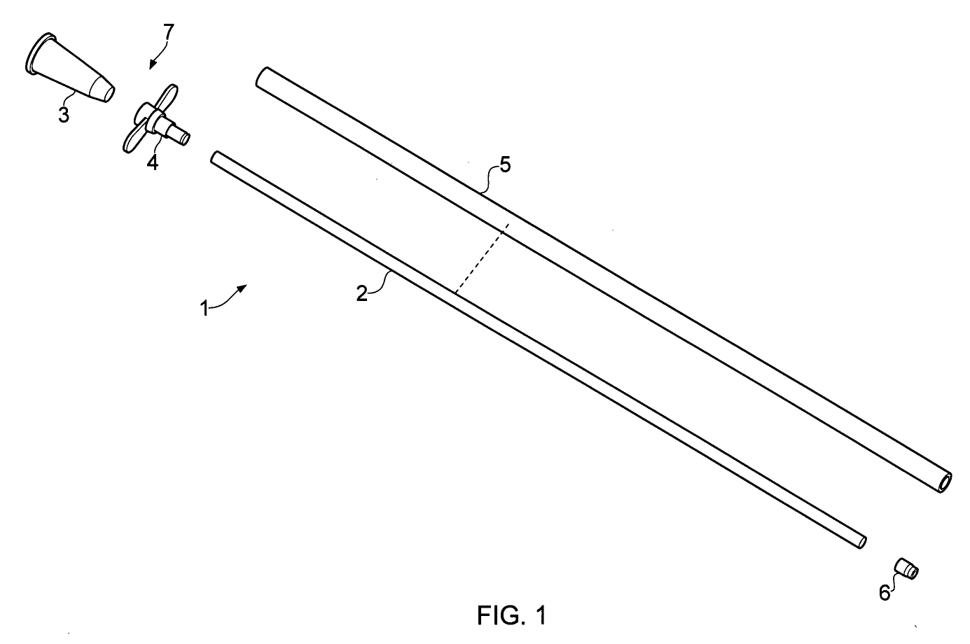

A sheath assembly 1 shown in FIG. 1 comprises an elastic elongate tube 2; a connection device 7 comprising a resilient fitting 3 and a connector 4; a rigid cover 5; and a cap 6.

The tube is manufactured by a process of extrusion as follows.
1. A batch of pellets of thermoplastic polyurethane is mixed with a batch of pellets of slip agent.
2. The mixture of pellets is fed into an extrusion chamber.
3. The extrusion chamber contains a helical worm screw which pushes the pellets along the length of the extrusion chamber.
4. The pellets are progressively heated as they move along the chamber, causing them to melt and become fully mixed.
5. The liquid mixture is then extruded from the extrusion chamber onto a pin and die tool. The pin and die tool has a pin running along its axis, and a cylindrical die surrounding the pin and spaced from it to form an annular extrusion chamber.
6. The mixture is extruded along the annular extrusion chamber and progressively cools. Thus the outer surface of the pin defines an inner diameter of the tube 2, and the inner surface of the die defines its outer diameter.
7. Once the tube has fully cooled, it is cut to size.

An example of a suitable mixture is:
90% by weight of Estane (R) 58311 Nat 022, supplied by Lubrizol Corporation. This is an aromatic polyether-based themoplastic polyurethane, provided in spherical pellet form; and
10% by weight of Estane (R) T5318 NAT 019, supplied by Lubrizol Corporation. This is an aromatic, polyether TPU based, mould release masterbatch, provided in cylindrical pellet form.

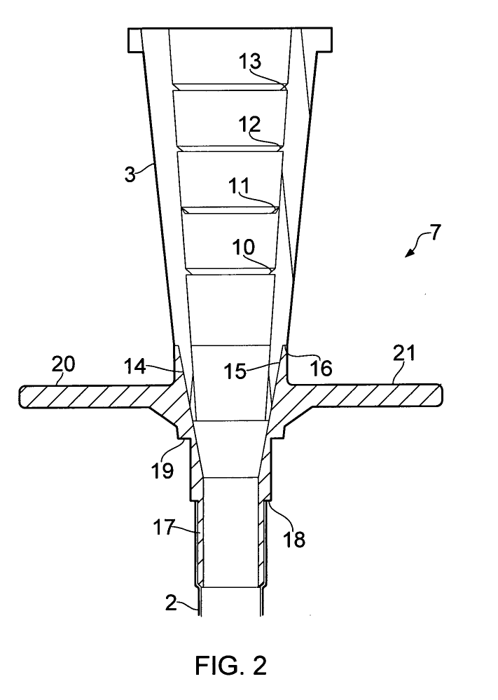

The connection device 7 formed by the combination of the fitting 3 and connector 4 is shown in cross-section in FIG. 2. The fitting 3 is formed by injection moulding as a single piece from a flexible resilient elastomeric material such as VYT/11X-65 RV1 CLEAR TL 0001. This is a Vythene compound, supplied by AlphaGary Corporation, which combines the best qualities of thermoplastic elastomer and vinyl at an economical cost. Alternatively a soft PVC (with a Shore A hardness less than 60) may be used.

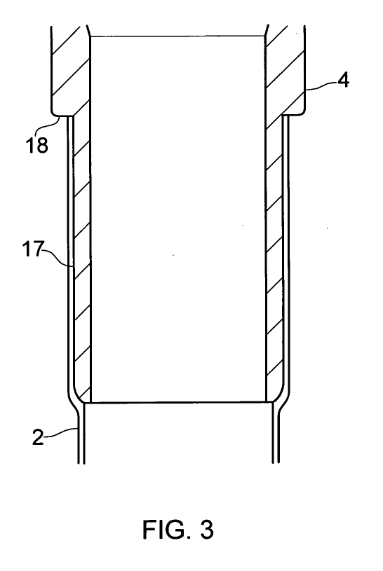

The connector 4 is formed by injection moulding as a single piece from a rigid material such as BASF Terlux 2802 TR MABS. This is an acrylonitrile butadiene styrene (ABS) thermoplastic polymer. The connector 4 comprises a collar with a proximal end attached to the fitting 3 and a distal end attached to the tube 2, as shown in further detail in FIG. 3.

The connector 4 is attached to the tube 2 and fitting 3 by the following process.
1. The tapered distal end 14 of the fitting 3 is dipped into methylcyclohexanone solvent which forms a thin film on the dipped surfaces of the fitting.
2. The tapered distal end 14 of the fitting 3 is pushed into the tapered proximal end of a bore 15 running through the connector 4 until the connector 4 engages an annular shoulder 16.
3. The methylcyclohexanone dissolves and fuses together the contacting surfaces, and evaporates from any other surfaces.
4. After step 3, the non-tapered distal end 17 of the connector 4 is dipped into methylcyclohexanone which forms a thin film on the dipped surfaces of the connector.
5. The proximal end of the tube 2 is fitted onto the non-tapered distal end 17 of the connector 4 until the tube 2 engages an annular shoulder 18. The solvent acts as a lubricant which makes it easy to slide the tube on, even if the inner diameter of the tube 2 is slightly smaller than the outer diameter of the distal end 17 (as is the case in FIG. 3). If necessary, a pair of jaws can be used to slightly open up the tube as it is pushed on. Note that the inner diameter of the tube 2 may be approximately the same as the outer diameter of the distal end 17, so little or no deformation of the tube is required in order to push it onto the connector.
6. The methylcyclohexanone dissolves and fuses together the contacting surfaces, and evaporates from any other surfaces.

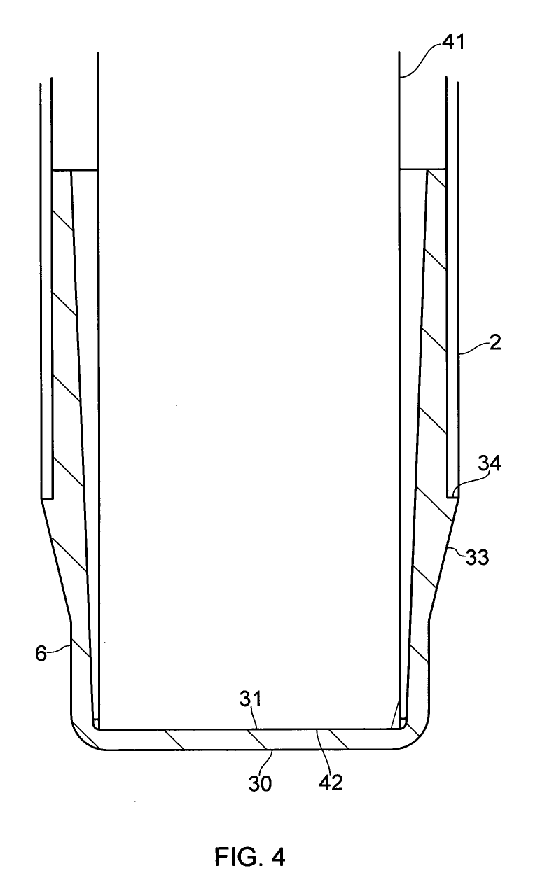

FIG. 4 shows the distal end of the assembly 1 in cross-section, with the endoscope insertion tube 41 in place. The cap 6 is formed by injection moulding as a single piece from a transparent material. The cap 6 has a transparent end wall with a pair of parallel circular faces 30,31, and a tubular body with a cylindrical inner surface 32 and an annular ridge 33 on its outer surface.

The cap 6 is attached to the tube 2 by the following process.
1. The proximal end of the cap 6 is dipped into methylcyclohexanone solvent which forms a thin film on the dipped surfaces of the cap.
2. The tube 2 is pushed onto the proximal end of the cap 6 until it engages a shoulder 34 of the annular ridge 33. Note that the inner diameter of the tube 2 is matched with the outer diameter of the cap 6 so that little or no stretching of the tube is required as it is pushed onto the cap.
3. The methylcyclohexanone dissolves and fuses together the contacting surfaces, and evaporates from any other surfaces.

The cover 5 is then slid up the tube 2 until it engages an annular shoulder 19 on the connector 4, as shown in FIG. 2. The assembly is then sterilised and packed in preparation for transport to a hospital.

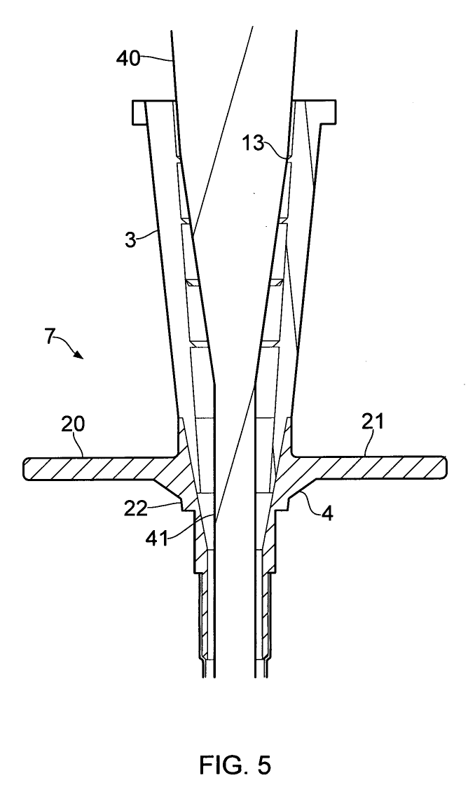

At the hospital, the assembly is unpacked and fitted to the endoscope by the following process, and as illustrated in FIG. 5.
1. The endoscope insertion tube 41 is inserted into the bore of the tube 2. The slip agent in the material forming the tube prevents the parts from sticking.
2. The connector 4 has a pair of wings 20, 21 which extending laterally from opposite sides of the connector. Due to the length of the fitting 3, these are positioned towards the distal end of the connection device 7 and thus prevent cross-infection with a tapered endoscope head 40. The resilient walls of the fitting 3 define a tapered bore for receiving and gripping the endoscope head 40 as shown in FIG. 5. The distal end of the connector 4 is gripped by a surgeon and then the wings 20,21 are pushed axially so as to fit the resilient fitting 3 over the endoscope head 40. The wings 20,21 protrude by approximately 12 mm from the outer wall 22 of the body of the connector, giving sufficient length to be easily pushed by a finger.
3. At some point (depending on the relative lengths of the insertion tube 41 and the tube 2) the distal end 42 of the endoscope insertion tube 41 engages the inner face 31 of the end cap 6.
4. Continued pushing of the wings causes the fitting 3 to move further onto the endoscope head, and also causes the tube 2 to stretch axially and press the inner face 31 of the end cap 6 tightly against the distal end 42 of the endoscope insertion tube 41. The amount of stretch of the tube may be relatively low (for example 2 mm) or high (for example 12 mm) depending on the relative lengths, although the tube 2 can be stretched by up to 50 mm without breaking if necessary.
5. The wings 20,2 are pushed until the endoscope head has become fully wedged into the tapered bore of the resilient fitting 3 and gripped by one or more annular ridges 10-13 in the bore. Depending on the degree of taper of the endoscope head 40, it may engage one or more of the annular ridges 10-13. For instance in FIG. 5 the head 40 engages only the upper one of the ridges 13
6. The cover 5 is then removed and the endoscope is ready for use.

Note that the connector 4 and the resilient fitting 3 are formed from different materials. The material forming the connector 4 has a higher elastic modulus than the material forming the resilient fitting 3. For instance BASF Terlux 2802 TR MABS has a tensile modulus of 2 GPa (290080 psi) and VYT/11X-65 RV1 CLEAR TL 0001 has a modulus at 100% elongation of 3.4 MPa (500 psi) As a result, the resilient fitting 3 is sufficiently flexible to enable it to easily expand laterally to accommodate the endoscope head. This makes the assembly easy to fit onto the endoscope head. The higher stiffness of the connector 4 has three advantages. Firstly, it enables the connector 4 to transmit axial forces evenly and efficiently to the fitting 3. Secondly, the wings 20,21 can be pushed without a significant degree of bending. Thirdly, it prevents the distal end 17 of the connector 4 from contracting laterally if the tube 2 is stretched onto it. Thus the bore through the connector 4 remains open and does not obstruct the passage of the endoscope insertion tube 41.

The lateral wings 20, 21 provide two advantages. Firstly, they provide a convenient and ergonomic surface to engage the fingers of the surgeon, making it easier to fit the assembly onto the endoscope head. Secondly, they acts as finger guards which keep the surgeon's fingers well away from the proximal end of the fitting 3, reducing the chance of cross-infection with the endoscope head 40.

Although the invention has been described above with reference to one or more preferred embodiments, it will be appreciated that various changes or modifications may be made without departing from the scope of the invention as defined in the appended claims.

1. A sheath assembly for protecting an endoscope insertion tube projecting from an endoscope head, the sheath assembly comprising:
a resilient elongate tube comprising a bore for receiving the endoscope insertion tube;
a resilient fitting for gripping the endoscope head; and
a connector comprising a proximal end attached to the resilient fitting and a distal end attached to the tube,
wherein the connector and the resilient fitting are formed from different materials, the material forming the connector having a higher elastic modulus than the material forming the resilient fitting.

2. The assembly of claim 1 wherein the resilient fitting is formed from a single piece of material.

3. The assembly of claim 1 wherein the connector is formed from a single piece of material.

4. The assembly of claim 1 wherein the connector comprises one or more laterally extending projections.

5. The assembly of claim 4 wherein the laterally extending projections comprise a pair of wings extending laterally from opposite sides of the connector.

6. The assembly of claim 4 wherein the projection(s) are positioned towards the proximal end of the connector.

7. The assembly of claim 1 wherein the distal end of the connector is received in a proximal end of the bore of the tube.

8. The assembly of claim 7 wherein the distal end of the connector has an outer surface which engages an inner surface of the tube and is substantially non-tapered.

9. The assembly of claim 1 wherein the connector comprises a bore, and the resilient fitting comprises a distal end which is received in a proximal end of the bore of the connector.

10. The assembly of claim 1 wherein the resilient fitting comprises a tapered bore for receiving and gripping the endoscope head.

11. The assembly of claim 1 wherein the resilient fitting comprises an elastomeric material.

12. The assembly of claim 1 wherein the tube comprises a slip agent.

13. The assembly of claim 1 wherein the tube is extruded.

14. The assembly of claim 1 wherein the tube comprises an elastomeric material.

15. The assembly of claim 1 wherein the assembly is adapted such that when the resilient fitting is fitted onto the endoscope head, the elastic elongate tube stretches axially and presses against a distal end of the endoscope insertion tube.

16. A method of protecting an endoscope insertion tube projecting from an endoscope head using the assembly of claim 1, the method comprising:
inserting the endoscope insertion tube into the bore of the resilient elongate tube;
gripping the connector; and
applying a force to the connector so as to push the resilient fitting onto the endoscope head.

17. The method of claim 16 wherein the force is applied to one or more laterally extending projections.

18. The method of claim 16 wherein the application of axial force to the connector causes the elastic elongate tube to stretch axially and press against a distal end of the endoscope insertion tube.

19. A sheath assembly for protecting an endoscope insertion tube projecting from an endoscope head, the sheath assembly comprising:
a resilient elongate tube comprising a bore for receiving the endoscope insertion tube; and
a connection device attached to a proximal end of the tube, the connection device comprising a resilient fitting for gripping the endoscope head; and a pair of wings extending laterally from opposite sides of the connection device.

20. The assembly of claim 20 wherein the wings are positioned towards a distal end of the connection device.

21. A method of protecting an endoscope insertion tube projecting from an endoscope head using the assembly of claim 19, the method comprising:
inserting the endoscope insertion tube into the bore of the resilient elongate tube; and
applying a force to the wings so as to push the resilient fitting onto the endoscope head.
